(19) United States
(12) Patent Application Publication
(10) Pub. No.: US 2024/0201235 A1
(43) Pub. Date: Jun. 20, 2024

(54) SENSING SYSTEM AND ELECTRICITY METER ASSEMBLY

(71) Applicant: Aclara Meters LLC, St. Louis, MO (US)

(72) Inventors: Scott David Brown, North Conway, NH (US); Christopher Andrew Porter, Weare, NH (US)

(73) Assignee: Aclara Meters LLC, St. Louis, MO (US)

(21) Appl. No.: 18/541,290

(22) Filed: Dec. 15, 2023

Related U.S. Application Data
(60) Provisional application No. 63/433,248, filed on Dec. 16, 2022.

(51) Int. Cl. G01R 22/00; G01N 33/00

(52) U.S. Cl. CPC G01R 22/00; G01N 33/0063

(57) ABSTRACT

A utility metering device includes a housing positioned in communication with a utility distribution system. A utility sensor unit is configured to measure at least one parameter of the utility distribution system to obtain utility system data. A gas sensor unit is in communication with an external environment outside of the housing and configured to obtain gas data related to the air quality of the external environment. One or more processors are in communication with the utility sensor unit and the gas sensor unit. A memory unit is in communication with the one or more processors. A communication unit is configured to send and receive data over a network. A sensing system can be created using a plurality of meters to detect environmental conditions.

### RELATED APPLICATION(S)

This application is based on Provisional Application Ser. No. 63/433,248, filed Dec. 16, 2023, the disclosure of which is incorporated herein by reference in its entirety and to which priority is claimed.

### FIELD

The present disclosure relates to sensing systems and utility meter assemblies, and more particularly, implementing gas sensors within a network of utility meters, for example electricity meters.

### BACKGROUND

Utility grids carry utilities (e.g., gas, electric, water, etc.) from generation or distribution facilities to end users in various locations throughout the world. Meters are placed throughout these grids to monitor and record distribution data. Connected or smart utility meters are increasingly common in utility systems. These connected meters allow for data to be provided over a network to a central utility system for processing, billing, maintenance, etc.

One example is the use of smart meters in electrical distribution systems. Smart meters can be connected to an electrical grid to monitor electrical distribution data in the system. These meters can be powered directly form the grid to eliminate the need for external power sources, which can boost the operability time and communication abilities over the meters, especially in remote locations.

### SUMMARY

According to certain implementations, a utility metering device includes a housing positioned in communication with a utility distribution system. A utility sensor unit is configured to measure at least one parameter of the utility distribution system to obtain utility system data. A gas sensor unit is in communication with an external environment outside of the housing and configured to obtain gas data related to the air quality of the external environment. One or more processors are in communication with the utility sensor unit and the gas sensor unit. A memory unit is in communication with the one or more processors. A communication unit is configured to send and receive data over a network.

According to certain implementations, an electrical metering device includes a housing positioned in communication with an electrical distribution system. An electrical sensor unit is configured to measure at least one parameter of the electrical distribution system to obtain electrical system data. A gas sensor unit is in communication with an external environment outside of the housing and configured to obtain gas data related to the air quality of the external environment. One or more processors are in communication with the electrical sensor unit and the gas sensor unit. A memory unit is in communication with the one or more processors. A communication unit is configured to send and receive data over a network.

According to certain implementations, a sensing system includes a plurality of electricity meters. Each electricity meter includes a memory unit, one or more processors, a communication unit configured to communicate over a network, an electrical sensor unit configured to measure at least one parameter of an electrical distribution system, and a gas sensor. The gas sensor is in communication with the processor and is configured to provide gas sensor data to the processor. A remote monitor is in communication with the plurality of meters over the network. At least one electricity meter of the plurality of electricity meters is configured to communicate the gas sensor data over the network to the remote monitor. The remote monitor is configured to be accessed by a user device.

According to certain implementations, a method of determining environmental conditions from a utility meter includes providing a utility meter along a utility distribution system. The utility meter includes a utility sensor unit configured to measure at least one parameter associated with the utility distribution system and a gas sensor unit in communication with an external environment. The gas sensor unit obtains gas sensor data associated with the air quality of the external environment. The gas sensor data is transmitted to a remote monitor via a network.

According to certain implementations, an electrical metering device includes a housing positioned in communication with an electrical distribution system. An electrical sensor unit is configured to measure at least one parameter of the electrical distribution system to obtain electrical system data. A gas sensor unit is in communication with an external environment outside of the housing and configured to obtain gas data related to the air quality of the external environment. One or more processors are in communication with the electrical sensor unit and the gas sensor unit. The one or more processors is configured to implement a neural network to analyze the gas sensor data to determine if an alarm condition is met. A memory unit is in communication with the one or more processors. A communication unit is configured to send and receive data over a network.

According to certain implementations, an electrical metering device includes a housing positioned in communication with an electrical distribution system. An electrical sensor unit is configured to measure at least one parameter of the electrical distribution system to obtain electrical system data. A gas sensor unit is in communication with an external environment outside of the housing and configured to obtain gas data related to the air quality of the external environment. One or more processors are in communication with the electrical sensor unit and the gas sensor unit. The one or more processors is configured to process the gas sensor data to reduce the dimensionality of the data A memory unit is in communication with the one or more processors. A communication unit is configured to send and receive data over a network.

The disclosure herein should become evident to a person of ordinary skill in the art given the following enabling description and drawings. The drawings are for illustration purposes only and are not drawn to scale unless otherwise indicated. The drawings are not intended to limit the scope of the invention. The following enabling disclosure is directed to one of ordinary skill in the art and presupposes that those aspects within the ability of the ordinarily skilled artisan are understood and appreciated.

### BRIEF DESCRIPTION OF THE DRAWINGS

Various aspects and advantageous features of the present disclosure will become more apparent to those of ordinary skill when described in the detailed description of preferred embodiments and reference to the accompanying drawings.

FIG. 1 shows a schematic representation of an illustrative embodiment of a sensing system.

FIG. 2 shows a schematic representation of an illustrative embodiment of an electricity meter assembly.

FIG. 3 shows an illustrative process whereby a remote monitor configures an electricity meter to control or otherwise communicate with a gas sensor.

FIG. 4 shows an illustrative process whereby a remote monitor retrieves gas measurement data from an electricity meter.

FIG. 5 shows an example of a heater profile.

FIG. 6 shows an illustrative process whereby the dimensionality of gas sensor data is reduced.

FIG. 7 shows an exemplary plot of gas sensor data after dimensionality reduction.

FIG. 8 shows an illustrative process whereby a neural network is trained to analyze gas sensor data to determine an alert condition.

FIG. 9 shows an example of an Air Quality Index (AQI) reference table.

FIGS. 10 and 11 show partially exploded views of an illustrative embodiment of an electricity meter assembly.

FIG. 12 shows an assembled view of the illustrative electricity meter assembly of FIGS. 10 and 11, without the cover and hydrophobic mesh.

### DETAILED DESCRIPTION

Various embodiments are directed to systems, methods, and apparatus for providing gas monitoring services utilizing a utility meter. Air quality monitoring has become an important task. It can be used to determine pollutants in the air and also to identify hazardous conditions, such as fires or gas leaks. Utilizing a utility meter with a gas monitoring sensor can provide a number of advantageous over a specialized, dedicated networks of sensors deployed to gather air quality data.

In certain embodiments, a power meter may be provided, and the power meter may facilitate communication with one or more monitoring systems and user devices, such as personal computers and/or mobile devices. For example, the power meter may communicate data and that data can be viewed in a certain format on a user device. The data may be provided to a user device through a web portal or other application. Additionally, various user commands associated with the operation of the power meter and/or gas monitoring sensor can be transmitted to the meter to adjust or control the operation of the device.

Data from the meters can be processed to determine the occurrence of an event and alerts or notifications can be provided to a user as needed. A user can also be presented with one or more graphical representations of real-time or historical data obtained from one or more meters. Utilizing such a system can help to monitor air quality, monitor gas leaks, detect fires, and to determine the start point or initiation of a hazardous condition such as a fire.

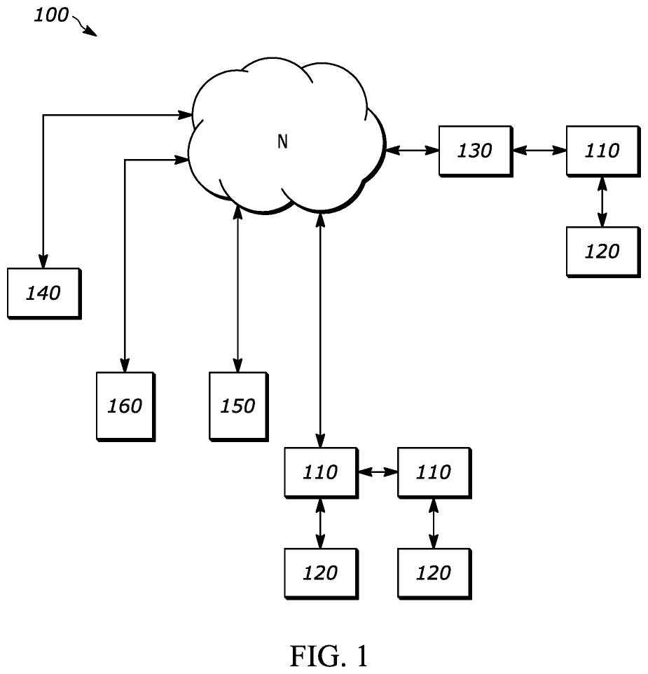

FIG. 1 shows an illustrative embodiment of a sensing system 100 comprising a plurality of utility meters, for example electricity meters 110 configured to communicate over a network N. Each electricity meter 110 can include or be in communication with a gas sensor 120. Each meter 110 can be configured to communicate over the network N or certain meters 110 can be configured to communicate with a meter 110 that is connected to a network. In certain configurations, a meter 110 can have a communication link to a gateway 130 and/or other devices with communication capability for accessing the network N. These communications can include different wired or wireless communications and utilize any type of communication protocol as would be understood by one of ordinary skill in the art. Although electricity meters 110 are shown, the systems and components described herein can be used with other utility meters, including gas, water, etc.

In certain configurations the system can include a remote monitor 140 connected to the network to receive data from the meters 110, including gas monitoring data. The remote monitor 140 can include a server having a communication interface, memory, and one or more processors. The remote monitor 140 can receive and store data from the meters 110, process the data, and provide an output.

A user device 150 can be connected to the network N. The user device 150 can be a personal computer, mobile device, tablet, or other computing device that is configured to access the network N and transmit and receive data through the network N to the remote monitor 140 and/or the meters 110. In certain implementation the user device 150 can be directly or locally connected to the remote monitor 140, so that communication over the network is not required. Various combinations of user devices can be used.

One or more servers 160 can also be connected to the network N for storing data from the meters 110 and/or the remoter monitor 140. The server 160 can also be configured to handle communications and the exchange of data packets between the devices. One of ordinary skill will understand that fewer components can be used or additional components can be incorporated into the system 100 as needed.

Each electricity meter 110 can be connected to an electrical grid and be configured to measure data from the electrical grid and receive data from the gas sensor 120. The received data can be processed by the meter 110 and communicated over the network N or raw data can be communicated from the meter 110 over the network N. The data can be communicated to any combination of the remote monitor 140, the user device 150, and the server 160.

The gas sensor 120 can include, but is not limited to, a micro electro-mechanical system (MEMS) gas sensor. Potential advantages of such a configuration can include a small footprint, low cost, and low power consumption. Examples of suitable gas sensors 120 can include, but are not limited to, BME688 (commercialized by Bosch Sensortec®) or ZMOD4510 (commercialized by Renesas®). The gas sensor 120 can be implemented as a gas sensor on a printed circuit board (PCB). The BME688 is a gas sensor that utilizes a metal-oxide (MOX) surface. The electrical resistance of the MOX surface is affected in part by the composition of the gas around the surface and by the temperature of the surface.

Providing gas sensing capabilities at the electricity meters 110 can provide a number of advantages. For example, utilizing an already deployed and powered network of electricity meters 110 can reduce the cost, labor, time, and complexity compared to implementing stand-alone gas sensors over a large geographical region. Powering gas sensors 120 at the meter 110 to the electrical grid can also help avoid the need for batteries or alternate sources of power. Such sustained power can enable the gas sensors 120 to be operable for a longer period of time, and to transmit data over larger distances, compared to conventional stand-alone devices which are limited by virtue of having to conserve power.

Furthermore, using meters 110 to perform gas sensing over a large geographical area can provide accurate detection of hazardous incidents such as smoke or wildfires, and can help identify the source of such incidents quickly and in greater detail than previously achieved. Detailed air quality maps can also be generated, e.g., in real time. When used with programmable gas detectors, the sensing system 100 can further help detect gasoline fumes, natural gas leaks, methane sources, electrical fires, propane leaks, sewer gas, carbon monoxide leaks, and other hazardous incidents.

Providing gas sensing capabilities at the electricity meters 110 can provide additional synergetic benefits. For example, electricity meters 110 are typically more densely distributed in more highly populated areas. This is particularly advantageous when detecting events that are more likely to occur in highly populated areas, such as certain types of fires or certain types of gas leaks.

The remote monitor 140 can be a remote device that is bidirectionally connected, via the network N, to the electricity meters 110. The communication of the network N can be operable to communicate data packets over the network N. The network N can include any combination of devices and wired and/or wireless communication links. Feeding into the network N are electricity meters 110, which can be residential electricity meters, commercial electricity meters, or a combination thereof. The remote monitor can be a utility or another entity.

The network N can be an advanced metering infrastructure (AMI) network. In some embodiments, the network can be a cellular network. In certain configurations, the network N can employ radio data transmission. For example, lower frequency (e.g., about 450 MHZ) transmission can be utilized for communication. Compared to higher frequency (e.g., 900 MHZ) signals, a lower frequency signal can better penetrate obstacles and propagate more effectively, and can travel considerably further using the same power level in the transmitter. This is especially advantageous when meters are located in meter pits or basements or behind other obstacles, such as dense tree forests.

The network N can also offer distributed data collection involving redundant coverage, and using existing electric infrastructure, for example, by employing data collection units (DCUs) mounted on existing power poles and structures. As such, each meter can be read by multiple DCUs, thus leading to desirable redundancy. Furthermore, using existing electric infrastructure can result in lower power and operational costs, and facilitate management and repair efforts, compared to conventional systems which might utilize separate communication for gas sensors.

The electricity meters 110 can include a communication component (e.g., a transceiver) configured for communication via the network N. The communication component can be operable to communicate data packets using a variety of protocols over different communication media, such as wired and/or wireless communication. Examples of communication technologies and/or protocols can include LoRaWAN, WiFi-ah (802.11ah), 3G, 4G, LTE, 5G, Sigfox, Ingenu, Digimesh, Synergize RF, or TWAC PLC, Bluetooth low energy, Bluetooth mesh networking, near-field communication, Thread, TLS (Transport Layer Security), Wi-Fi (e.g., IEEE, 802.11), Wi-Fi Direct (for peer-to-peer communication), Z-Wave, Zigbee, HaLow, cellular communication, LTE, low-power wide area networking (Sigfox, Lora, Ingenu), VSAT, Ethernet, MoCA (Multimedia over Coax Alliance), PLC (Power-line communication), DLT (digital line transmission), etc. Other suitable communication technologies and/or protocols can be used without deviating from the scope of the present disclosure. In certain implementations LPWAN protocols can provide increased range of communication. HaLow can provide lower energy requirements and higher data rates. Other protocols can be used without deviating from the scope of the present disclosure.

In some embodiments, the network N can include a local area network and/or a wide area network. In some embodiments, the network N can include one or more of a secure network, Wi-Fi network, AMI network, mesh network, the Internet, cellular network, or other wide area network, one or more peer-to-peer communication links, and/or some combination thereof, and can include any number of wired or wireless links. Communication over the network N can be accomplished, for instance, via a communication interface using any type of protocol, protection scheme, encoding, format, packaging, etc.

The computing systems and devices discussed herein can include one or more processors and one or more memory devices. The computing systems and devices can be distributed such that its components are located in different geographic areas. The technology discussed herein refers to computer-based systems and actions taken by, and information sent to and from, computer-based systems. One of ordinary skill in the art will recognize that the inherent flexibility of computer-based systems allows for a great variety of possible configurations, combinations, and divisions of tasks and functionality between and among components. For instance, processes discussed herein may be implemented using a single computing device or multiple computing devices working in combination. Databases, memory, instructions, and applications may be implemented on a single system or distributed across multiple systems. Distributed components may operate sequentially or in parallel.

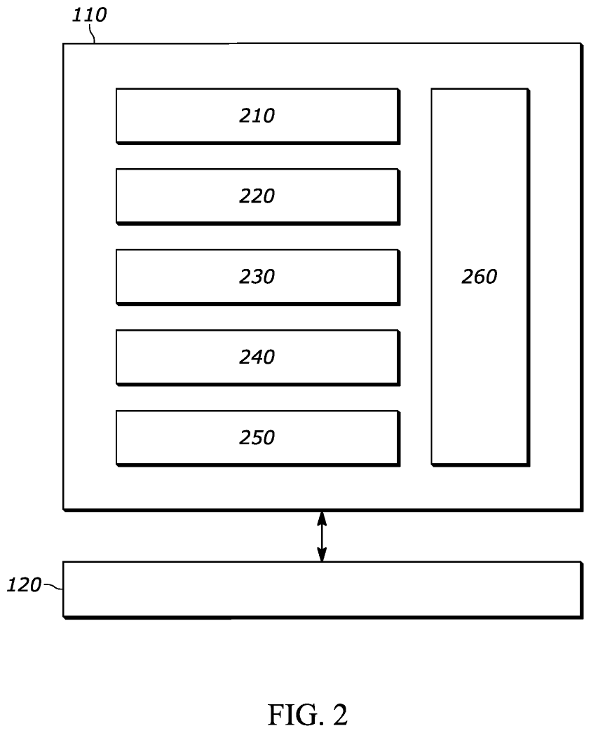

FIG. 2 shows an exemplary configuration of the electricity meter 110. The electricity meter 110 can be configured to measure power consumption and used to report a variety of power quality measurements. The electricity meter 110 can include a power supply 210, one or more memory units 220, one or more processors 230, a clock unit 240, a communication unit 250, and an electrical sensor unit 260.

The power supply 210 is configured to provide power to the components of the meter 110. The power supply 210 can also provide power to the gas sensor 120 of the same electricity meter assembly. In some configurations, the power supply 210 can include a connection to an electrical grid. For example, the power supply 210 can include a transformer with its primary windings coupled to the incoming power distribution lines and having windings to provide a nominal voltage, e.g., 5 VDC, +12 VDC and −12 VDC, at its secondary windings. In other embodiments, power may be supplied from an independent power source to the power supply 210. For example, power may be supplied from a different electrical circuit or an uninterruptible power supply (UPS). In some embodiments, the power supply 210 can include an energy storage device, such as a battery and/or a capacitor. In some embodiments, the power supply 210 can include a power harvesting system that is configured to charge and/or store energy in the energy storage device for powering the electricity meter 110 and the gas sensor 120. The power harvesting system can be configured to harness for instance, one or more of solar energy, wind energy, piezoelectric energy, electromagnetic energy associated with power lines suspended by a utility pole, or radio frequency energy.

The electricity meter 110 can be equipped with one or more memory units 220. The memory units 220 can be configured to store configuration data and log data which can include real-time and historical data from the gas sensor 120 and the electrical sensor unit 260. The one or more memory units can contain any combination of volatile and non-volatile memory. The volatile memory can be internal storage memory such as random access memory. The non-volatile memory can include removable memory such as a solid-state storage memory, e.g., a CompactFlash card, a Memory Stick, SmartMedia card, MultiMediaCard (MMC), SD (Secure Digital) memory. The one or more processors 230 can be configured to write data into the memory 220.

The one or more processors 230 can include, for instance, microcontrollers, microprocessors, logic circuits, application specific integrated circuits, etc. The processors 230 can be configured to perform calculations on the received data and to control the overall operation of the meter 110 and gas sensor 120.

The one or more memory units 220 can store computer-readable instructions that when executed by the one or more processors 230 cause the one or more processors to provide functionality according to example aspects of the present disclosure. For instance, the memory 220 can store computer-readable instructions that when executed by the one or more processors 230 cause the one or more processors 230 to implement any of the data processing techniques and/or communication techniques disclosed herein. Furthermore, the data processing techniques and/or communication techniques disclosed herein can be implemented by one or more processors, such as locally by one or more processors at the electricity meter 110, at a remote device (e.g., a remote monitor 140, a user device 150 or a cloud server 160), or can be shared across multiple devices (e.g., a plurality of electricity meters 110, remote monitors 140, user devices 150, cloud servers 160, etc.). Those of ordinary skill in the art, using the disclosure provided herein, will understand that various steps of any of the methods provided herein can be adapted, modified, rearranged, performed simultaneously, include steps not illustrated, or expanded in various ways without deviating from the scope of the present disclosure.

In some embodiments, the electricity meter 110 can include a clock 240, such as a real-time clock. The clock 240 can be used, for instance, to associate timestamp data with the data obtained by (or derived from the output of) the various sensors associated with the electricity meter 110, including but not limited to electricity sensors 260 and/or the gas sensor 120. The timestamp data can be used, for instance, to perform historical processing, identify trends, identify times associated with event occurrences, for comparison to other utility poles, and other purposes.

The clock 240 can be set during installation of the electricity meter 110. Various methods can be used to address clock drift (e.g., shifting of time provided by clock 240 relative to true time). For instance, the clock 240 can periodically sync with time data from remote devices when sending and/or receiving communications.

The communication unit 250 enables communication between the meter 110 and an external device such as another meter or other computer device over a remote and/or local network. The communication unit 250 can be a modem, network interface card (NIC), wireless transceiver, etc. The communication unit 250 performs its functionality by hardwired and/or wireless connectivity. The hardwire connection may include but is not limited to hard wire cabling e.g., parallel or serial cables, RS232, RS485, USB cable, Firewire (1394 connectivity) cables, Ethernet, and the appropriate communication port configuration. The wireless connection will operate under any of the various wireless protocols including but not limited to Bluetooth™ interconnectivity, infrared connectivity, radio transmission connectivity including computer digital signal broadcasting and reception commonly referred to as Wi-Fi or 802.11.X (where x denotes the type of transmission), satellite transmission or any other type of communication protocols, communication architecture or systems currently existing or to be developed for wirelessly transmitting data including spread spectrum 900 MHZ, or other frequencies, Zigbee, WiFi, or any mesh enabled wireless communication.

The electrical sensor unit 260 can include one or more sensors to sense electrical parameters of the grid, e.g., voltage and current on incoming power lines. The sensor unit 260 can also include one or more A/D converters. For example, the sensors can include current transformers and voltage transformers, wherein one current transformer and one voltage transformer is coupled to each phase of the incoming power lines. A primary winding of each transformer will be coupled to the incoming power lines and a secondary winding of each transformer will output a voltage representative of the sensed voltage and current. The output of each transformer can be coupled to an A/D converter configured to convert the analog output voltage or current to a digital signal that can be processed by the one or more processors 230 and stored in memory 220.

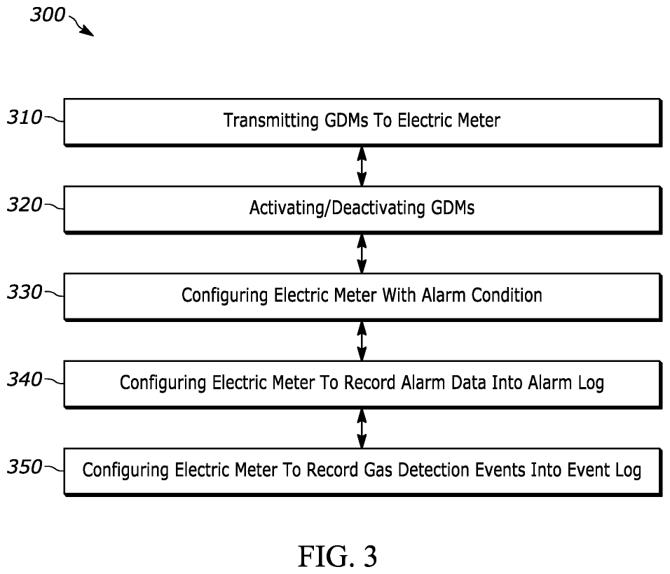

FIG. 3 shows an example of a process 300 whereby the electricity meter 110 is configured to control the gas sensor 120 located at the electricity meter 110. The gas sensor 120 can be programmed or reprogrammed to operate under certain parameters to detect one or more external conditions. This can be triggered by an external device, such as the remote monitor 140 or the user device 150 The configuration subprocesses described below can be executed in various orders.

In an exemplary step 310, a device transmits one or more gas detection module (GDMs), via the network N, to the electricity meter 110. A GDM can include configuration settings for detecting specific gases or gas combinations. The GDM can be stored in the memory 220.

In certain configurations, the GDM can include a heater profile for the gas sensor associated with an optimized configuration to detect a certain environmental condition. For example, different gas sensors 120 can detect gases utilizing a resistance measurement of a surface, such as a MOX resistance as previously described. Because the temperature of the MOX surface can affect the measurement, different temperature profiles can more accurately detect different gases. Accordingly, the heater profile can include instructions to heat the MOX surface to one or more specific temperatures over a set period of time. For example, the surface can be heated to 300 degrees Celsius for one second, then the temperature dropped to 100 degrees Celsius for six seconds, then raised to 200 degrees Celsius for two seconds, and then raised to 300 degrees Celsius for two seconds. Different combinations of temperatures and times can be used for a specific heater profile to provide the optimal sensing conditions for different gases.

In an exemplary step 320, a device can selectively activate and/or deactivate the GDMs. This selection can be determined by a user desiring to detect certain gas or gas combinations. Alternatively, this selection can be algorithmically determined by a computer program having information about the likelihood of certain gas or gas combinations being present at the location of the electricity meter 110. For example, if the computer program determines such likelihood to be above a predetermined threshold, then the computer program can be configured to activate one or more GDMs corresponding to the detection of such gas or gas combinations. The threshold can also be identified using machine learning techniques or other processing techniques. Such event occurrence can be identified when there is a threshold crossing. The magnitude of the threshold crossing can be indicative of the amount that the data exceeds and/or falls below a threshold.

In an exemplary step 330, the electricity meter 110 can be configured to provide a notification or alert when a predetermined alarm condition is met. For example, if a gas or gas combination is detected by the gas sensor 120 to be present in excess of a predetermined amount or concentration, an alert or notification can be sent out over the network N. The predetermined amount or concentration of gas or gas combination may be a function of a historical baseline, such as an average of historical gas or gas combination amounts or concentration values detected at the electricity meter 110, or may be a function of gas or gas combination amounts or concentration values detected at nearby electricity meters 110. The predetermined amount or concentration can also be identified using machine learning techniques or other processing techniques. Such event occurrences can be identified when there is a threshold crossing. The magnitude of the threshold crossing can be indicative of the amount that the data exceeds and/or falls below a threshold.

In some embodiments, the alarm can be provided by the remote monitor 140 in a graphical user interface presented on a display screen associated with the remote monitor 140 or a remote device associated therewith (such as the user device 150) or can be provided in the form or other output (e.g., audio, tactile, etc.). A graphical user interface can present other information associated with data collected by one or more electricity meters 110, such as reports, comparisons, charts, analytics, etc.

In an exemplary step 340, the electricity meter 110 can be configured, via the network N, to record alarm data into an alarm log stored in the memory 220. For example, the alarm log can include alarm events associated with a predetermined alarm condition being met. For example, alarm events can include a timestamp and a description of the condition being met.

In an exemplary step 350, the electricity meter 110 can be configured, via the network N, to record gas detection events into an event log stored in the memory 220. For example, gas detection events can include measurements performed at predetermined times, such as at regular intervals or preset dates/times, and/or can include measurements yielding a gas amount or concentration in excess of a predetermined value, or a classification of gasses considered abnormal or dangerous. For example, the event log can include a timestamp and a result of the measurement made.

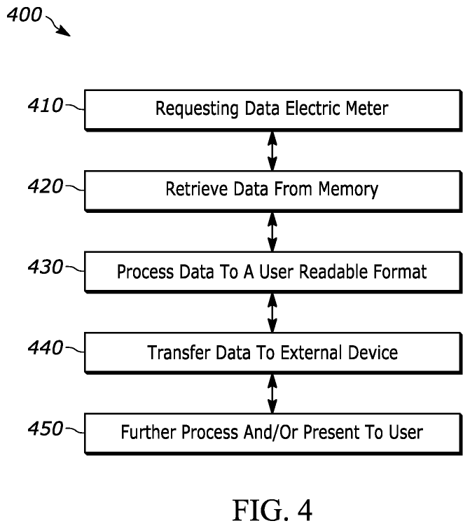

FIG. 4 shows an example of a process 400 whereby a device such as the remote monitor 140, user device 150, or server 160 can retrieve gas measurement data from the electricity meter 110 and present the retrieved data to a user. The retrieval subprocesses described below can be executed in various orders, sequentially or simultaneously.

In an exemplary step 410, data is requested from the electricity meter 110. The data can include an event log, an alarm, log, real-time data, or other data. The requested data is retrieved from memory in step 420. If necessary, the data can be processed to a user readable format. This processing can be completed by the one or more processors 230 in the meter 110 or by the external device. In certain configurations, the data can be processed to a user readable format prior to being stored in memory. The data can be transferred to an external device in step 440 to be presented to a user and/or to be further processed by the device in step 450. For example, the further processing of the data can include providing a graphical representation of current and past data to a user. This graphical representation can include a map showing air quality data over a given region and at a certain time or time frame. The information can be color coded to provide a visual representation to the user.

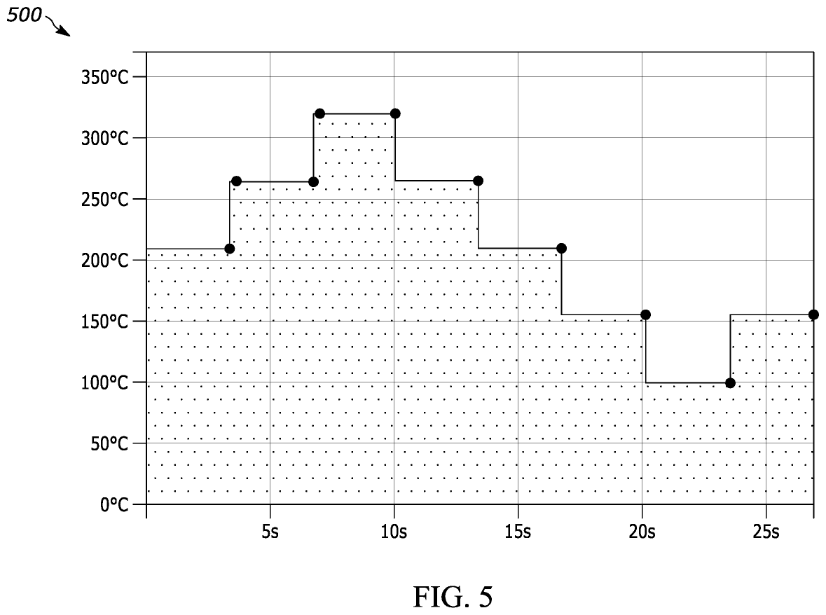

In certain configurations, the electricity meter 110 can be configured to processes the gas sensor data prior to storage or transmission to reduce the amount of data that needs to be stored or transmitted. For example, sensor data can be obtained in different steps through a single heater profile. FIG. 5 shows an example of a heater profile, with the points representing the steps at which data is gathered by the sensor. Each step is a single dimension of data that is obtained, resulting in ten dimensions of raw data.

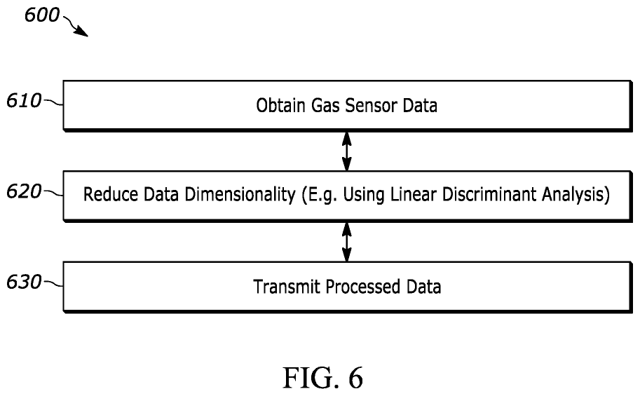

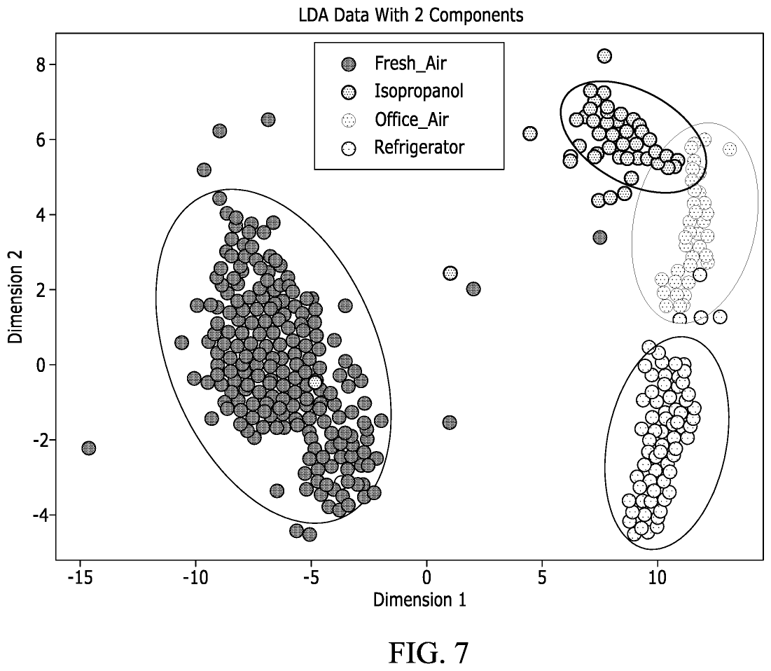

FIG. 6 shows an example of a process 600 performed on the data to simplify the amount of data for storage and transmission while retaining accurate results. Data is obtained from the gas sensor in step 610. The data can represent different dimensions of data obtained from the gas sensor. After the data is obtained, the dimensionality of the data is reduced in step 620. The dimensionality of the data can be reduced by applying one or more algorithms to the data to process the data into fewer dimensions. In an exemplary configuration, linear discriminant analysis (LDA) can be used to process the data. LDA can be used to find linear combinations of features that separate two or more classes of data and form the data into clusters. FIG. 7 shows the resultant data that can be obtained after gas sensor data is processed. After the data is processed the processed data can be transmitted in step 630. Further actions can then be taken with the processed data, including being transmitted to memory for storage, compiled into a log, added to an alert, or transmitted to a device such as a remote monitor or other server. In various implementations, processing the data 600 to reduce the dimensionality is performed by one or more processors 230 in the electricity meter 110.

In certain configurations, the electricity meter 110 can be configured to utilize a neural network to determine if a predetermined alarm condition is met. A neural network can be trained to analyze the sensor data to detect specific conditions indicative of an alarm condition, such as the present of a sufficient amount of smoke over a period of time to indicate a fire. The neural network can be trained in a similar fashion to what is described in U.S. Published Application No. 2023/0341477, the disclosure of which is hereby incorporated by reference in its entirety.

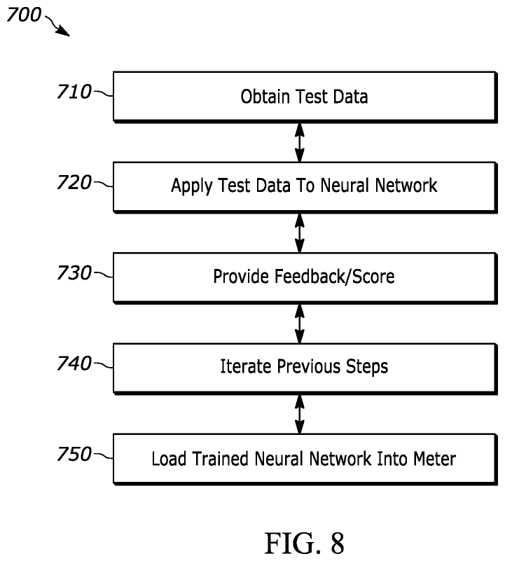

For example, the neural network may be a multi-layer perceptron neural network that utilizes a number of hidden nodes to analyze input data from the gas sensor and classify the data to determine if an alarm condition is met. FIG. 8 shows an exemplary process 700 for training a neural network and applying it to gas sensor data obtained by the electricity meter 110. Test data is obtained in step 710 for use in training the neural network. The test data can be obtained, for example, through laboratory testing under controlled conditions using the relevant gas sensor units 120 operating under certain heater profiles. The test data is applied to the neural network at step 720 and the output from the network is scored and feedback provided in step 730. Steps 710-730 can be iterated as necessary to obtain satisfactory confidence in the model as shown in step 740. In certain implementations, several gas detectors can be run simultaneously to detect different gases and the results can be compiled to provide different packages to a plurality of meters in the field.

In some implementations, the trained neural network can then be loaded into the meter and used to analyze data obtained from the gas sensor 120. In some implementations the neural network data can be updated and loaded into the field in real time over the network. The analysis can be performed on raw data or on processed data. In certain configurations the neural network is loaded into the memory of the meter and the one or more processors are configured to implement the neural network to analyze the gas sensor data to determine the alarm condition is met.

In operation, the electricity meter 110 can configure the gas sensor 120 based on configuration data stored in memory 220 of the electricity meter 110. The configuration data can include data from or generated using the GDM, such as, for example, but not limited to, configuration data setting a frequency of gas sensor measurements, or configuration data setting a sensing profile for the gas sensor 120. A sensing profile can include, for example, a pre-programmed temperature profile configured for making an air quality measurement, based on, for example, temperature, as well as barometric pressure, humidity, and/or any other parameters known to influence sensor measurements.

The gas sensor 120 can periodically (e.g., at predetermined intervals), send gas measurements to the electricity meter 110. Those gas measurements can be interpreted and/or stored in the memory 220.

The above data relating to gas measurements can be communicated, via the network N, in conjunction with data obtained by the electricity meter 110 relating to electricity measurements, such as, but not limited to, electric consumption data. Collecting and/or communicating electricity data together with gas measurement data can advantageously provide, for example, valuable data when detecting an electrical fire. For example, the gas measurement data could be used to detect a gas or gas composition associated with electrical fires, while the electrical data could be used to determine an electrical event associated with electrical fires (e.g., an electrical short). Accordingly, these data can advantageously help confirm a determination that an electrical fire has occurred. In such a case, a remote disconnecting function can be utilized to remotely shut off an electrical circuit connected to the electricity meter.

Data communication over the network N can be performed by sending one or more data packets from the electricity meter 110 to the remote monitor 140. A data packet can include, for example, a header, a payload, and a verification portion. The payload can include an identifier of the electricity meter 110, such as, for example, a serial number and/or other identifier associated with a particular electricity meter 110. The identifier of the electricity meter 110 can assist remote devices, such as the remote monitor 140, in determining where a particular event occurrence is located (e.g., by coordinating the identifier of the electricity meter 110 with a location stored in a database). The payload can include data associated with a condition of the electricity meter 110, such as data derived from the result of a measurement performed by the electrical sensor unit 260 or the gas sensor 120. The data can be transmitted after processing, conditioning and/or filtering by one or more processors 230 of the electricity meter 110. Data packets transmitted to the electricity meter 110 from the remote monitor 140 can likewise include an identifier of the electricity meter 110.

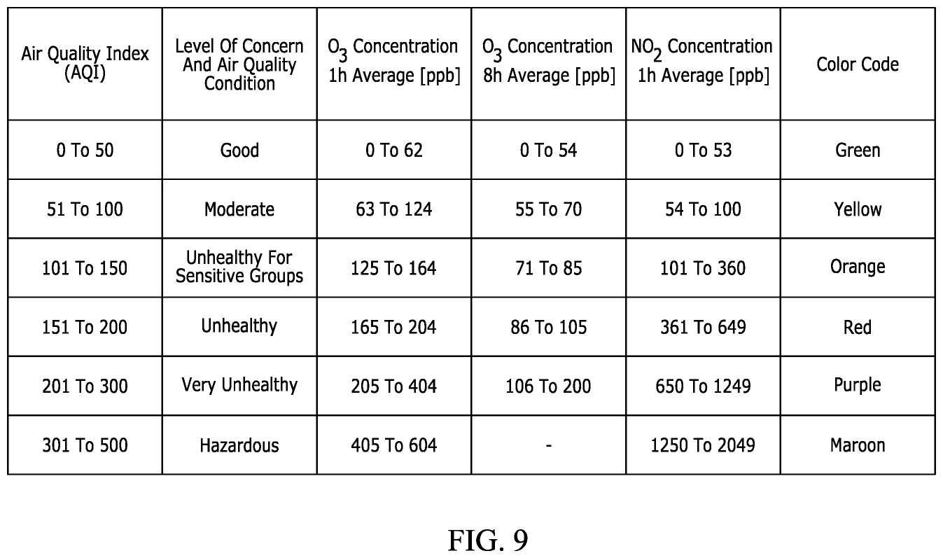

In some embodiments, output by the sensing system 100 can be in the form of data indicative of air quality. For example, a graphical user interface (e.g., at the remote monitor 140 or the user device 150), can display a color indicative of air quality, or any other information, for example with reference to the Air Quality Index (AQI) reference table shown in FIG. 9. Furthermore, the sensing system 100 can create a detailed map (e.g., a real-time map) of air quality over a geographical region. This is particularly advantageous as various regulatory authorities (e.g., the United States Environmental Protection Agency) have recently demonstrated specific interest in improving air quality sensing.

In some embodiments, output by the sensing system 100 can be indicative of smoke or wildfire detection, or greenhouse gas detection. In some embodiments, output by the gas sensors 120, and thus by the sensing system 100, can be indicative of measurements of any of: temperature, barometric pressure, humidity, ozone and nitrogen dioxide, carbon dioxide, particulates (e.g., MP2.5 and PM10, which conventionally require complex and large sensors), or custom gas combinations. Detection of custom gas combination can be programmed, and machine learning can assist in identifying desired gas combinations.

In some configurations, the gas sensors 120 can include metal-oxide (MOX) sensors. A MOX sensor can detect gases by oxidation/reduction on its sensitive layer. Metal-oxide changes resistance due to chemical reaction (with reduction corresponding to lower resistance and oxidation corresponding to higher resistance) and reacts to most volatile compounds and pollution gases. The sensor temperature affects oxidation and reduction. Accordingly, heater temperature and duration can be programmed, with target temperatures typically between 200° ° C. and 400° C. Specific gas compositions can be identified, by performing multiple resistance measurements at different temperatures and durations. Machine learning can be can be used to identify unique compositions as discussed herein.

In some configurations, the gas sensors 120 can include a plurality of sensors that are installed as a module into the meter 110. The sensors can include MOX, sensors, MEM sensors, particle matter sensors, and other sensors.

In some embodiment, the sensing system 100 can utilize programmable sensor heating profiles to perform AQI measurements or identify a gas composition. For example, Herrmann et al., Air Quality Measurement Based on Advanced PM2.5 and VOC Sensor Technologies, Sensors & Transducers, Vol. 243, Issue 4, August 2020, pp. 1-5 (https://sensorsportal.com/sensors_and_transducers.html) (incorporated herein by reference in its entirety) FIG. 5 illustrates how environmentally sensed data, compared with reference data, can enable the detection of different gas compositions.

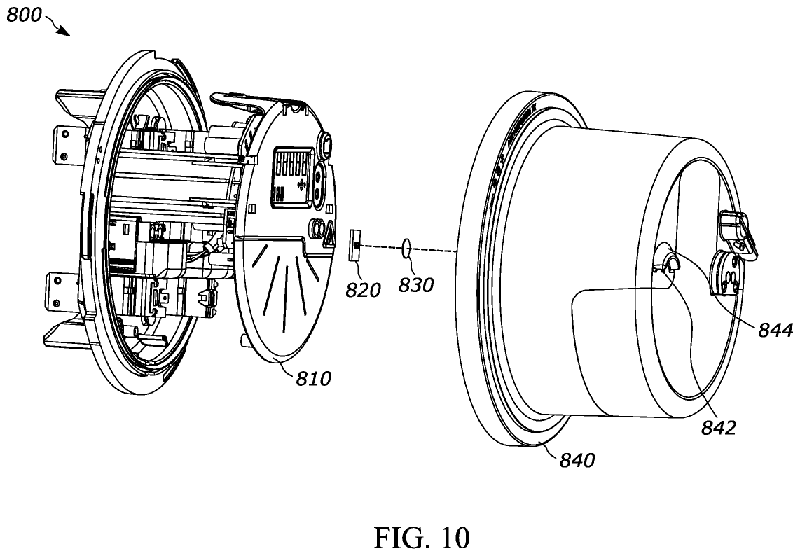

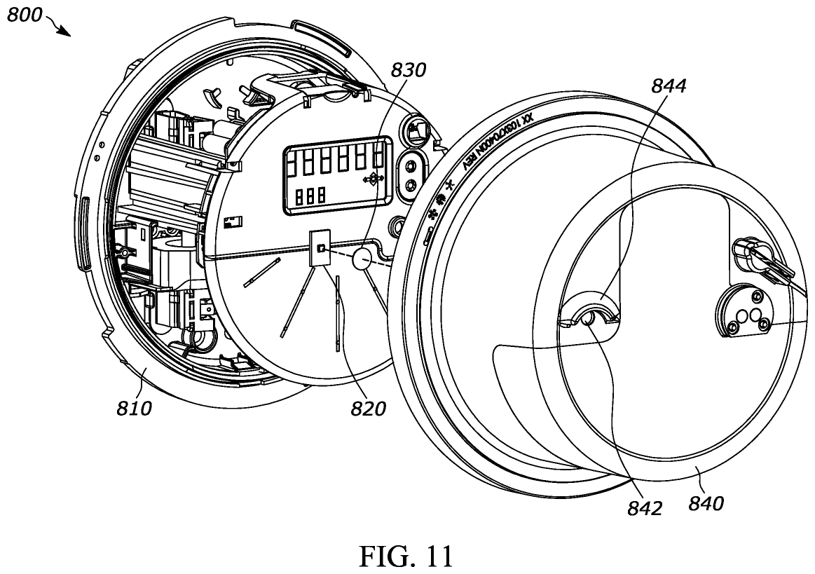

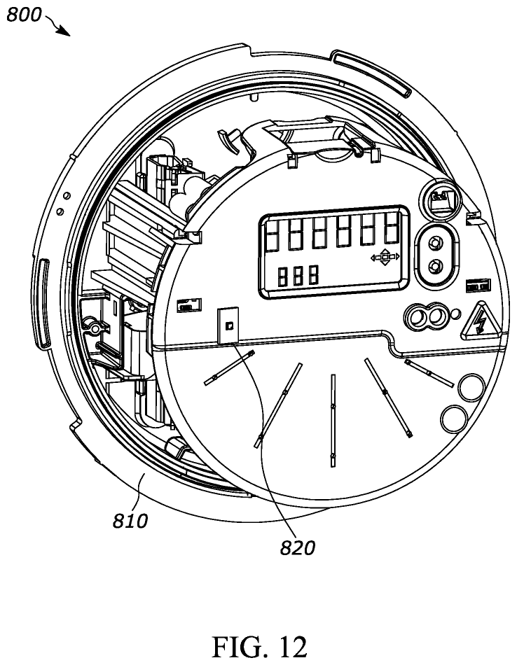

FIGS. 10-12 show an illustrative embodiment of an electricity meter assembly 800, including an electricity meter 810 and a gas sensor 820. The gas sensor 820 can be attached to the electricity meter 810. For example, the gas sensor 820 can be mounted onto or with the electricity meter 810.

In some embodiments, a removable or fixed cover 840 of the electricity meter assembly 800, which forms part of its housing, protects components of the electricity meter assembly 800 from the elements. The cover 840 can include an opening 842 providing communication between the gas sensor 820 and the external environment. In some embodiments, the opening 842 is located on a portion of the housing of the electricity meter assembly 800 other than its cover 840.

One technical challenge in implementing a gas sensor 820 in electricity meter 810 is that, on the one hand, the gas sensor 820 should be exposed to the elements to allow for sufficient airflow for the detection of gases, but on the other hand, the electricity meter 810 should be protected from the elements for safety reasons. This technical challenge can be addressed in the following manner according to illustrative embodiments of the present disclosure. For example, the electricity meter assembly 800 can further include a hydrophobic mesh 830 positioned between the opening 842 and the gas sensor 820, for protecting the gas sensor 820 from the elements. In addition, the cover 840 can include an overhang 844 protecting the opening 842 from the elements.

One of ordinary skill will appreciate that the exact dimensions and materials are not critical to the disclosure and all suitable variations should be deemed to be within the scope of the disclosure if deemed suitable for carrying out the objects of the disclosure.

One of ordinary skill in the art will also readily appreciate that it is well within the ability of the ordinarily skilled artisan to modify one or more of the constituent parts for carrying out the various embodiments of the disclosure. Once armed with the present specification, routine experimentation is all that is needed to determine adjustments and modifications that will carry out the present disclosure.

The above embodiments are for illustrative purposes and are not intended to limit the scope of the disclosure or the adaptation of the features described herein, for example, to particular electricity meter assemblies or gas sensors. Those skilled in the art will also appreciate that various adaptations and modifications of the above-described preferred embodiments can be configured without departing from the scope and spirit of the disclosure. Therefore, it is to be understood that, within the scope of the appended claims, the invention may be practiced other than as specifically described.

What is claimed is:

1. An electrical metering device comprising:
a housing positioned in communication with an electrical distribution system;
an electrical sensor unit configured to measure at least one parameter of the electrical distribution system to obtain electrical system data;
a gas sensor unit in communication with an external environment outside of the housing and configured to obtain gas data related to the air quality of the external environment;
one or more processors in communication with the electrical sensor unit and the gas sensor unit;
a memory unit in communication with the one or more processors; and
a communication unit configured to send and receive data over a network.

2. The electrical meter device of claim 1, further comprising a power supply configured to provide power to the gas sensor.

3. The electrical meter device of claim 2, wherein the power supply receives power from the electrical distribution system.

4. The electrical meter device of claim 1, wherein the one or more processors are configured to execute instructions to vary the temperature of a sensing surface of the gas sensor over a time interval.

5. The electrical meter device of claim 1, wherein the one or more processors are configured to process the gas data to reduce the dimensionality of the data.

6. The electrical meter device of claim 1, wherein the one or more processors are configured to implement a neural network to analyze the gas data to determine if an alarm condition is met.

7. The electrical meter device of claim 1, wherein the housing includes an opening providing communication between the gas sensor and the external environment.

8. The electrical meter device of claim 7, wherein a hydrophobic mesh is positioned between the gas sensor and the opening.

9. The electrical meter device of claim 7, wherein the housing includes an overhang extending outwardly above the opening.

10. A sensing system comprising:
a plurality of electricity meters, each electricity meter including a memory unit, one or more processors, a communication unit configured to communicate over a network, an electrical sensor unit configured to measure at least one parameter of an electrical distribution system, and a gas sensor, wherein the gas sensor is in communication with the processor and is configured to provide gas sensor data to the processor; and
a remote monitor in communication with the plurality of meters over the network, wherein at least one electricity meter of the plurality of electricity meters is configured to communicate the gas sensor data over the network to the remote monitor,
wherein the remote monitor is configured to be accessed by a user device.

11. The sensing system of claim 10, wherein at least one electricity meter of the plurality of electricity meters includes a power source configured to provide power to the associated one or more processors and the gas sensor.

12. The sensing system of claim 10, wherein the one or more processors of at least one electricity meter of the plurality of electricity meters is configured to process the gas sensor data to reduce the dimensionality of the data.

13. The sensing system of claim 10, wherein the remote monitor is configured to transmit one or more gas detection modules, via the network, to the one or more electricity meters of the plurality of electricity meters.

14. The sensing system of claim 13, wherein the gas detection module includes a heater profile.

15. The sensing system of claim 10, wherein the remote monitor is configured to selectively activate and/or deactivate a gas detection module stored in one or more electricity meters of the plurality of electricity meters.

16. The sensing system of claim 10, wherein one or more electricity meters of the plurality of electricity meters are configured to alert the remote monitor when a predetermined alarm condition is met.

17. The sensing system claimed in claim 16, wherein the one or more processors of the plurality of electricity meters is configured to implement a neural network to analyze the gas sensor data to determine if the alarm condition is met.

18. The sensing system claimed in claim 10, wherein one or more electricity meters of the plurality of electricity meters is configured to record gas detection events into an event log stored in the memory of the electricity meter.

19. The sensing system claimed in claim 10, wherein the remote monitor is configured to retrieve the gas sensor date from the plurality of electricity meters.

20. A method of determining environmental conditions from a utility meter comprising:
providing a utility meter along a utility distribution system, the utility meter having a utility sensor unit configured to measure at least one parameter associated with the utility distribution system and a gas sensor unit in communication with an external environment;
obtaining, via the gas sensor unit, gas sensor data associated with the air quality of the external environment; and
transmitting the gas sensor data to a remote monitor via a network.
